(19) United States
(12) Patent Application Publication
(10) Pub. No.: US 2026/0047996 A1
(43) Pub. Date: Feb. 19, 2026

(54) COMPOSITION OF WATER-IN-SILICONE TYPE THAT BECOMES TRANSPARENT AT REFRIGERATED TEMPERATURES

(71) Applicant: AMOREPACIFIC CORPORATION, Seoul (KR)

(72) Inventor: Dongwan HA, Yongin-si (KR)

(73) Assignee: AMOREPACIFIC CORPORATION, Seoul (KR)

(21) Appl. No.: 19/275,549

(22) Filed: Jul. 21, 2025

(30) Foreign Application Priority Data
Aug. 13, 2024 (KR) 10-2024-0108233

(51) Int. Cl. A61K 8/891; A61K 8/06; A61Q 19/00

(52) U.S. Cl. CPC A61K 8/891; A61K 8/064; A61Q 19/00; A61K 2800/10

(57) ABSTRACT

The present disclosure relates to a water-in-silicone composition that is transparent at refrigerated temperatures, and more particularly, the composition includes: a water phase; and a silicone phase, wherein the difference in refractive index between the water phase and the silicone phase at room temperature is 0.003 to 0.004, the composition is opaque at room temperature and transparent at refrigerated temperatures, allowing the temperature of the composition to be visually recognized, and furthermore, the composition can provide a cool and refreshing feeling of use in sensation not only to the touch but also to the sight when used after being stored refrigerated.

### CROSS REFERENCE TO RELATED APPLICATION

The present application claims priority to Korean Patent Application No. 10-2024-0108233, filed Aug. 13, 2024, the entire contents of which are hereby incorporated by this reference.

### BACKGROUND OF THE INVENTION

### Field of the Invention

The present disclosure relates to a water-in-silicone composition that becomes transparent at refrigerated temperatures.

### Description of the Related Art

Emulsion cosmetic products are widely used as cosmetics because they can implement the advantages of both oil and water phases. Meanwhile, in order to improve the appearance of emulsion cosmetic compositions and satisfy the aesthetic sense of consumers, there have been many attempts to develop a transparent emulsion cosmetic composition through various methods such as a refractive index matching method. Studies in the related art have focused mainly on developing cosmetics that are transparent at room temperature.

Meanwhile, there has been a growing consumer demand for cosmetic products that require refrigerated storage/distribution (for example: cosmetics containing raw materials that are active at low temperatures, such as antioxidants, whitening agents, and skin regenerators, cooling cosmetics, and the like). In response to such consumer demand, the present inventors have conducted research and development into cosmetics that become transparent at refrigerated temperatures.

### SUMMARY OF THE INVENTION

One aspect of the present disclosure is to provide a water-in-silicone composition that is opaque at room temperature and transparent at refrigerated temperatures.

One aspect of the present disclosure is to provide a composition for refrigerated storage, refrigerated distribution, or application to the skin at refrigerated temperatures.

A water-in-silicone composition according to one aspect of the present disclosure includes: a water phase; and a silicone phase, wherein the difference in refractive index between the water phase and the silicone phase at room temperature is 0.003 to 0.004, and the composition becomes opaque at room temperature and transparent at refrigerated temperatures.

A composition for refrigerated storage, refrigerated distribution, or application to the skin at refrigerated temperatures according to one aspect of the present disclosure includes the water-in-silicone composition.

The water-in-silicone composition according to one aspect of the present disclosure is opaque at room temperature and transparent at refrigerated temperatures. Due to these characteristics, the temperature of the composition can be visually recognized. Furthermore, when the composition is used after being stored refrigerated, it can provide a user with a cool and refreshing feeling of use in sensation not only to the touch but also to the sight.

The water-in-silicone composition according to one aspect of the present disclosure can be applied to cooling products, products requiring low temperature storage, distribution and/or use, or the like.

It can be visually recognized whether a product to which the water-in-silicone composition according to one aspect of the present disclosure is applied has been appropriately stored at refrigerated temperatures. At the same time, the product to which the water-in-silicone composition is applied becomes transparent when used after being appropriately stored at refrigerated temperatures, and can give a user a cool and refreshing feeling of use in sensation not only to the touch but also to the sight.

### BRIEF DESCRIPTION OF THE DRAWINGS

FIG. 1 is a photograph of the compositions of some Examples at room temperature and at refrigerated temperatures.

FIG. 2 illustrates a photograph of the compositions of an Example and a Comparative Example at room temperature and at refrigerated temperatures.

FIG. 3 illustrates a photograph of the compositions of an Example and a Comparative Example at room temperature and at refrigerated temperatures.

### DETAILED DESCRIPTION OF THE INVENTION

It should be understood that various exemplary embodiments of the present document and the terms used therein are not intended to limit the technical features set forth herein to particular exemplary embodiments and include various changes, equivalents, or replacements for a corresponding exemplary embodiment.

The present inventors developed a composition that is opaque at room temperature and becomes transparent at refrigerated temperatures.

One aspect of the present disclosure provides a water-in-silicone (W/S) composition that is opaque at room temperature and transparent at refrigerated temperatures.

The composition according to one aspect of the present disclosure changes in transparency between room temperature and refrigerated temperatures, and is transparent particularly at refrigerated temperatures. Due to these characteristics, the temperature of the composition may be visually recognized by a user, and when the composition is used after being stored refrigerated, it is possible to provide the user with a visually refreshing and cool feeling of use.

In an embodiment, the room temperature is apparent to a person with ordinary skill in the art, and for example, the room temperature may be a temperature between 15° C. and 25° C. For example, room temperature may be in the range of 15° C. or higher, 16° C. or higher, 17° C. or higher, 18° C. or higher, 19° C. or higher, 20° C. or higher, 21° C. or higher, 22° C. or higher, 23° C. or higher, 24° C. or higher, 25° C. or higher, 15° C. or lower, 16° C. or lower, 17° C. or lower, 18° C. or lower, 19° C. or lower, 20° C. or lower, 21° C. or lower, 22° C. or lower, 23° C. or lower, 24° C. or lower, 25° C. or lower, or any combination thereof.

In an embodiment, the refrigerated temperatures is apparent to the person with ordinary skill in the art, and for example, the refrigerated temperatures may be a temperature between 2° C. and 8° C. For example, the refrigerated temperatures may be in the range of 2° C. or higher, 3° C. or higher, 4° C. or higher, 5° C. or higher, 6° C. or higher, 7° C. or higher, 8° C. or higher, 2° C. or lower, 3° C. or lower, 4° C. or lower, 5° C. or lower, 6° C. or lower, 7° C. or lower, 8° C. or lower, or any combination thereof.

In an embodiment, the water-in-silicone composition may include a water phase; and a silicone phase. The water-in-silicone composition according to one aspect of the present disclosure may be a water-in-silicone emulsion composition in which the water phase is emulsified in the silicone phase.

The silicone phase may also be expressed as an oil phase or a silicone oil phase.

In an embodiment, the difference in refractive index between the water phase and the silicone phase at room temperature may be 0.003 or more, or 0.003 to 0.004, but is not limited thereto. For example, the difference in refractive index between the water phase and the silicone phase at room temperature may be 0.003 or more, 0.003 to 0.005, 0.003 to 0.004, 0.003 to 0.0035, or 0.003 to 0.0034.

In an example, a W/S composition i) having a difference in refractive index between the water phase and the silicone phase of less than 0.003 at room temperature and ii) including a silicone-based compound with a phenyl group in the silicone phase was transparent at room temperature, but became opaque at refrigerated temperatures. Compositions that are opaque at refrigerated temperatures are less likely to provide a visually refreshing, cooler feeling of use to a user than compositions that are transparent at refrigerated temperatures.

Values specified as numerical values in the present disclosure may allow for an error range within 10%, 9%, 8%, 7%, 6%, 5%, 4%, 3%, 2%, 1%, 0.5%, 0.1%, 0.05%, or 0.01%.

One aspect of the present disclosure provides a water-in-silicone (W/S) composition, wherein the composition includes a water phase; and a silicone phase, the difference in refractive index between the water phase and the silicone phase at room temperature is 0.003 to 0.004, and the composition is opaque at room temperature and transparent at refrigerated temperatures. Since the water-in-silicone composition and the difference in refractive index between the water phase and the silicone phase have been described above, a detailed description thereof will be omitted.

The W/S composition in which the difference in refractive index between the water phase and the silicone phase at room temperature is 0.003 or more according to one aspect of the present disclosure may include a silicone-based compound having a phenyl group in the silicone phase to reduce the difference in refractive index between the water phase and the silicone phase at refrigerated temperatures (for example: 4° C.), and ultimately, the W/S composition that is opaque at room temperature may become transparent at refrigerated temperatures. This may be due to the fact that, compared to silicone-based compounds having only methyl groups without phenyl groups, a silicone-based compound having a phenyl group exhibit a greater degree of increase in refractive index than the water phase at refrigerated temperatures (for example: 4° C.).

In an embodiment, the silicone phase may include a first silicone-based compound having a phenyl group. The first silicone-based compound may have one or more phenyl groups. In an example, a W/S composition including a first silicone-based compound in which the difference in refractive index between the water phase and the silicone phase at room temperature is 0.003 or more and the silicone phase has a phenyl group has an effect in which the composition is opaque at room temperature and becomes transparent at refrigerated temperatures.

In an embodiment, the first silicone-based compound having a phenyl group may be at least one of a dimethicone having a phenyl group or a trimethicone having a phenyl group, but is not limited thereto.

In an embodiment, the first silicone-based compound having a phenyl group may be a silicone-based compound including a repeating unit having 1 to 3 phenyl groups, but is not limited thereto.

In an embodiment, the first silicone-based compound having a phenyl group may be at least one selected from the group consisting of phenyl trimethicone, diphenyl dimethicone, and diphenylsiloxy phenyl trimethicone, but is not limited thereto.

In an embodiment, the refractive index of the water phase is higher than the refractive index of the silicone phase, at room temperature.

In an embodiment, the water phase may include at least one selected from the group consisting of water, glycerin, and a metal salt, but is not limited thereto.

The metal salt may be at least one selected from the group consisting of magnesium sulfate and sodium chloride, but is not limited thereto.

In an embodiment, the silicone phase may further include at least one selected from the group consisting of a second silicone-based compound including a C1 to C5 alkyl group and a silicone surfactant, but is not limited thereto.

The second silicone-based compound may not include a phenyl group.

The second silicone-based compound may be at least one selected from the group consisting of methyl trimethicone and propyl trimethicone, but is not limited thereto.

The silicone surfactant may be polyglyceryl-3polydimethylsiloxyethyldimethicone, but is not limited thereto.

In an embodiment, the water-in-silicone composition may be a cosmetic composition, a food composition, an oral composition, a non-therapeutic composition, a non-therapeutic oral composition, a composition for external application, or a pharmaceutical composition, but is not limited thereto. For example, the composition may be a cosmetic composition or a composition for external application. For example, the composition for external application may be a composition for external application to the skin or a composition for external application to the hair.

In an embodiment, the water-in-silicone composition may not include a polyethylene glycol (PEG)-based surfactant. For example, the PEG-based surfactant may be at least one selected from the group consisting of PEG-6 caprylic/capric glycerides and PEG-8 caprylic/capric glycerides, but is not limited thereto.

In an embodiment, the water-in-silicone composition may not include a dimethicone having a viscosity of 100 cs or less. For example, the dimethicone having a viscosity of 100 cs or less may be at least one of dimethicone 6 cs and dimethicone 10 cs, but is not limited thereto. The viscosity may be a viscosity measured at 25° C.

In an embodiment, the temperature of the water-in-silicone composition may be visually confirmed, allowing a user to easily confirm whether the water-in-silicone composition has been stored refrigerated or distributed refrigerated.

In an embodiment, the water-in-silicone composition may be for application to the skin at refrigerated temperatures.

In an embodiment, the water-in-silicone composition may be applied to cooling cosmetics, and cosmetics including raw materials that require low temperature storage and/or low temperature distribution (for example: toners, serums, emulsions, and the like), but is not limited thereto. A product to which the water-in-silicone composition according to one aspect of the present disclosure is applied may provide a user with a cool and refreshing feeling of use in sensation not only to the touch but also to the sight when used after being stored refrigerated.

Further, one aspect of the present invention provides a composition for refrigerated storage, refrigerated distribution, or application to the skin at refrigerated temperatures, which includes the above-described water-in-silicone composition.

In an embodiment, the composition for refrigerated storage, refrigerated distribution, or application to the skin at refrigerated temperatures may be a cosmetic composition, a food composition, an oral composition, a non-therapeutic composition, a non-therapeutic oral composition, a composition for external application, or a pharmaceutical composition, but is not limited thereto. For example, the composition may be a cosmetic composition or a composition for external application. For example, the composition for external application may be a composition for external application to the skin or a composition for external application to the hair.

For example, the composition for refrigerated storage, refrigerated distribution, or application to the skin at refrigerated temperatures may be a cooling cosmetic composition, or a cosmetic composition including raw materials that require low temperature storage and/or low temperature distribution, but is not limited thereto.

In addition, one aspect of the present invention provides a use of the above-described water-in-silicone composition for preparing a cosmetic composition that is opaque at room temperature and transparent at refrigerated temperatures.

Furthermore, one aspect of the present invention provides a use of the above-described water-in-silicone composition for preparing a composition for refrigerated storage, refrigerated distribution or application to the skin at refrigerated temperatures.

Since the water-in-silicone composition, the refrigerated temperatures, and the like have been described above, a detailed description thereof will be omitted.

In an embodiment, the composition for refrigerated storage, refrigerated distribution or application to the skin at refrigerated temperatures may be a cosmetic composition.

Further, one aspect of the present invention provides a method for confirming whether a composition has been stored or distributed at refrigerated temperatures,
wherein the composition has a formulation of the above-described water-in-silicone composition, and
the method includes: determining that the composition has been stored or distributed at refrigerated temperatures when the composition is transparent.

In an embodiment, the composition may be a cosmetic composition, a food composition, an oral composition, a non-therapeutic composition, a non-therapeutic oral composition, a composition for external application, or a pharmaceutical composition, but is not limited thereto.

In an embodiment, there is an advantage in that it is possible to confirm whether the water-in-silicone composition is transparent by the naked eye.

In addition, one aspect of the present invention provides a method for preparing a cosmetic composition that is opaque at room temperature and transparent at refrigerated temperatures, the method including preparing the above-described water-in-silicone composition.

In an embodiment, the cosmetic composition that is opaque at room temperature and transparent at refrigerated temperatures may require refrigerated storage, may require refrigerated distribution, or may need to be applied to the skin at refrigerated temperatures.

Hereinafter, the present invention will be described in detail with reference to Examples for specifically describing the present invention. The following Examples are provided for illustrative purposes only to aid in the understanding of the present invention, and the scope and spirit of the present invention are not limited thereby.

### 1. Preparation of Water-In-Silicone Composition

A water-in-silicone composition was prepared using the components and composition ratios shown in Table 1 below. Specifically, the components shown in the silicone part in Table 1 were weighed and then pre-dispersed through homomixing (6000 rpm for 3 minutes) to prepare a silicone part composition. Furthermore, the components shown in the water part of Table 1 were weighed, then mixed and thoroughly dissolved to prepare a water part composition. The water part composition was added to the pre-dispersed silicone part composition, and then subjected to homomixing (6000 rpm for 3 minutes). Thereafter, deaeration was performed to prepare water-in-silicone compositions of the Examples and Comparative Examples (Examples 1 and 2 and Comparative Examples 1 to 4). The refractive index of the water part and the refractive index of the silicone part in Table 1 below were measured using an ATAGO ABBE refractometer.

TABLE 1

| | | | | | Comparative | Comparative | Comparative | Comparative |
|---|---|---|---|---|---|---|---|---|
| Classification | | | Example | Example | Example | Example | Example | Example |
| (unit wt %) | | | 1 | 2 | 1 | 2 | 3 | 4 |
| Water | Distilled | | 23 | 23 | 25 | 40.2 | 25.2 | 25.09 |
| part | water (D.I | | | | | | | |
| | water) | | | | | | | |
| | Magnesium | | 1 | 1 | 1 | — | — | 1 |
| | sulfate | | | | | | | |
| | Sodium | | — | — | — | — | — | 0.91 |
| | chloride | | | | | | | |
| | Glycerin | | 36 | 36 | 34 | 29.8 | 34.8 | 33 |
| Refractive index of | | | 1.4146 | 1.4146 | 1.4098 | 1.3879 | 1.4099 | 1.4111 |
| water part (at room | | | | | | | | |
| temperature) | | | | | | | | |
| Silicone | Methyl | | 25 | 25 | 25 | 25 | 25 | 25 |
| part | trimethicone | | | | | | | |
| | Silicone- | Phenyl | 5 | 5 | 5 | — | — | 5 |
| | based | trimethicone | | | | | | |
| | compound | Diphenylsiloxy | — | 5 | — | — | — | — |
| | having | phenyl | | | | | | |
| | phenyl group | trimethicone | | | | | | |
| | | Diphenyl | 5 | — | 5 | — | 5 | 5 |
| | | dimethicone | | | | | | |

| | | | | | | |
|---|---|---|---|---|---|---|
| Polyglyceryl-3 | 5 | 5 | 5 | 5 | 5 | 5 |
| polydimeth- | | | | | | |
| ylsiloxyeth- | | | | | | |
| yldimethicone | | | | | | |

| | | | | | | |
|---|---|---|---|---|---|---|
| Refractive index of | 1.4115 | 1.4113 | 1.4115 | 1.3914 | 1.4115 | 1.4115 |
| silicone part (at room | | | | | | |
| temperature) | | | | | | |
| Difference in | 0.0031 | 0.0033 | 0.0017 | 0.0035 | 0.0016 | 0.0004 |
| refractive index | | | | | | |
| between water part | | | | | | |
| and silicone part | | | | | | |

Examples 1 and 2 are compositions, in which the silicone part includes a silicone having a phenyl group, and the difference in refractive index between the water part and the silicone part corresponds to 0.003 to 0.004. Comparative Examples 1, 3, and 4 are compositions, in which the silicone part includes a silicone having a phenyl group, but the difference in refractive index between the water part and the silicone part is outside a range of 0.003 to 0.004 (a composition having a difference of approximately 0.0004 to 0.002). Comparative Example 2 is a composition in which the difference in refractive index between the water part and the silicone part corresponds to a range of 0.003 to 0.004, but the silicone part does not include a silicone having a phenyl group.

### 2. Evaluation of Transparency According to Temperature Change

The transparency of each of the Examples and Comparative Examples prepared in ‘1. Preparation of water-in-silicone composition’ above was confirmed at refrigerated temperatures and at room temperature. Specifically, each of the compositions of the Examples and Comparative Examples was left to stand at room temperature (25° C.) for 1 hour, and the transparency of each composition was visually confirmed after the composition reached room temperature (25° C.). In addition, each of the compositions of the Examples and Comparative Examples was stored refrigerated, and the transparency was visually confirmed after each composition reached the refrigerated temperatures (4° C.).

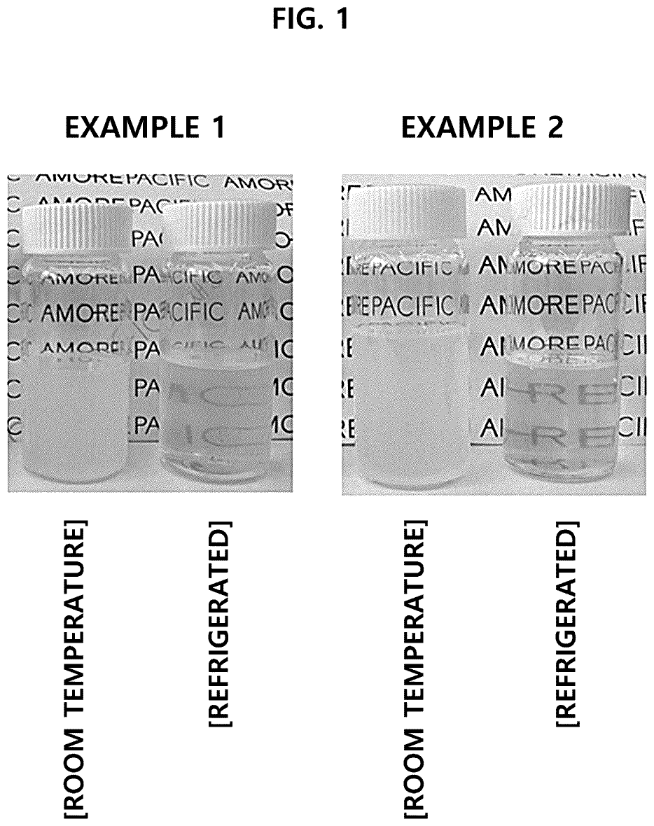

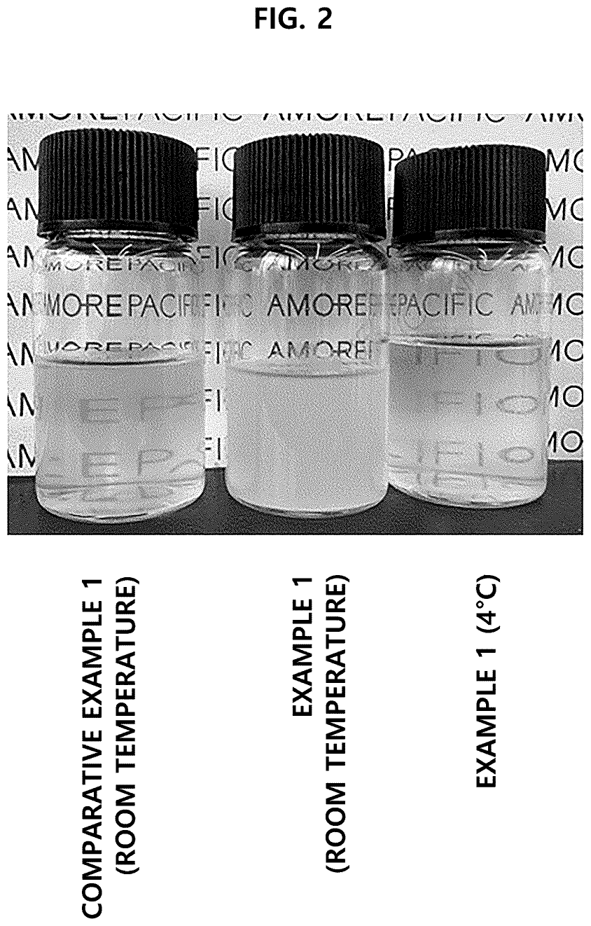

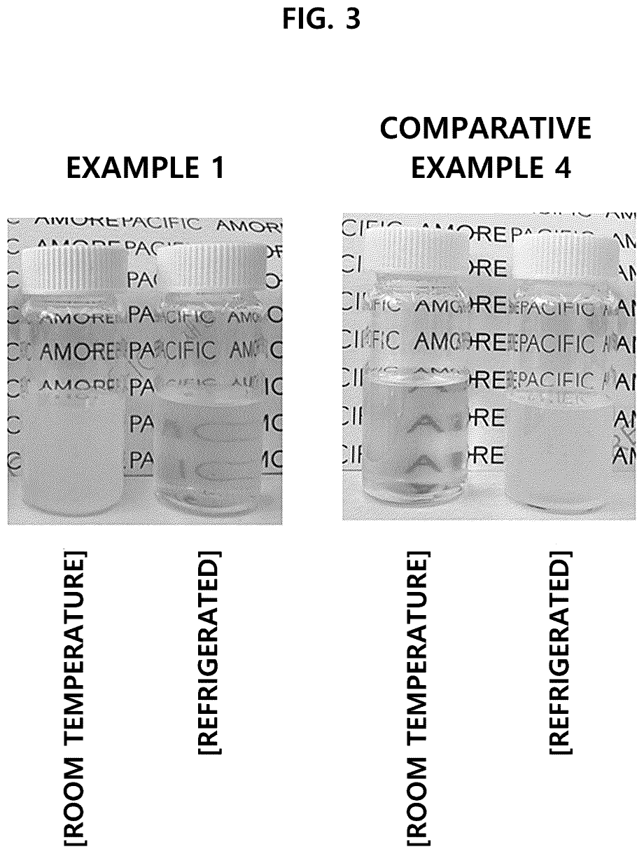

As a result, Examples 1 and 2, in which the silicone part includes a silicone having a phenyl group and the difference in refractive index between the water part and the silicone part is 0.003 to 0.004, were opaque at room temperature and transparent at refrigerated temperatures (FIGS. 1 and 2). Meanwhile, Comparative Example 4, in which the silicone part includes a silicone having a phenyl group, but the difference in refractive index between the water part and the silicone part is less than 0.003 to 0.004, was transparent at room temperature but became opaque at refrigerated temperatures (FIG. 3). Similarly to Comparative Example 4, Comparative Examples 1 and 3, in which the silicone part includes a silicone having a phenyl group, but the difference in refractive index between the water part and the silicone part is less than 0.003 to 0.004, were transparent at room temperature (FIG. 2). Meanwhile, Comparative Example 2, in which the difference in refractive index between the water part and the silicone part is 0.003 to 0.004, but the silicone part does not include a silicone having a phenyl group, was opaque at room temperature and remained opaque even at refrigerated temperatures without any change in transparency.

The compositions of the Examples are opaque at room temperature and become transparent at refrigerated temperatures, allowing the user to visually confirm the temperature. Furthermore, since the compositions of the Examples are transparent at refrigerated temperatures, the compositions may provide the user with a cool and refreshing feeling of use in sensation not only to the touch but also to the sight when used after being stored refrigerated.

What is claimed is:

1. A water-in-silicone (W/S) composition comprising:
a water phase; and a silicone phase,
wherein a difference in refractive index between the water phase and the silicone phase at room temperature is 0.003 to 0.004, and
the composition is opaque at room temperature and transparent at refrigerated temperatures.

2. The water-in-silicone composition of claim 1, wherein the silicone phase comprises a first silicone-based compound having a phenyl group.

3. The water-in-silicone composition of claim 2, wherein the first silicone-based compound having a phenyl group is at least one of a dimethicone having a phenyl group or a trimethicone having a phenyl group.

4. The water-in-silicone composition of claim 2, wherein the first silicone-based compound having a phenyl group is a silicone-based compound comprising a repeating unit having 1 to 3 phenyl groups.

5. The water-in-silicone composition of claim 2, wherein the first silicone-based compound having a phenyl group is at least one selected from the group consisting of phenyl trimethicone, diphenyl dimethicone, and diphenylsiloxy phenyl trimethicone.

6. The water-in-silicone composition of claim 1, wherein a refractive index of the water phase is higher than the refractive index of the silicone phase, at room temperature.

7. The water-in-silicone composition of claim 1, wherein the water phase comprises at least one selected from the group consisting of water, glycerin, and a metal salt.

8. The water-in-silicone composition of claim 1, wherein the silicone phase comprises at least one selected from the group consisting of a second silicone-based compound comprising a C1 to C5 alkyl group and a silicone surfactant.

9. The water-in-silicone composition of claim 8, wherein the silicone surfactant is polyglyceryl-3 polydimethylsiloxyethyldimethicone.

10. The water-in-silicone composition of claim 1, wherein the composition is a water-in-silicone cosmetic composition.

11. The water-in-silicone composition of claim 1, wherein the composition does not comprise a polyethylene glycol (PEG)-based surfactant.

12. The water-in-silicone composition of claim 1, wherein the composition does not comprise at least one of dimethicone 6 cs and dimethicone 10 cs.

13. The water-in-silicone composition of claim 1, wherein the composition is visibly identifiable as to whether it has been stored refrigerated or distributed refrigerated.

14. The water-in-silicone composition of claim 1, wherein the composition is for application to the skin at refrigerated temperatures.

15. A method for confirming whether a composition has been stored or distributed at refrigerated temperatures,
wherein the composition has a formulation of the water-in-silicone composition of claim 1, and
the method comprises: determining that the composition has been stored or distributed at refrigerated temperatures when the composition is transparent.

16. The method of claim 15, wherein the composition is a cosmetic composition.
